(19) United States
(12) Patent Application Publication
(10) Pub. No.: US 2016/0312169 A1
(43) Pub. Date: Oct. 27, 2016

(54) TEMPERATURE AND GAS CONTROLLED INCUBATOR

(71) Applicants: Michael Cecchi, Longboat Key, FL (US); Monica Mezezi, Guelph (CA); Timothy Schimmel, Randolph, NJ (US); Michael R. Cecchi, Longboat Key, FL (US)

(72) Inventors: Michael Cecchi, Longboat Key, FL (US); Monica Mezezi, Guelph (CA); Timothy Schimmel, Randolph, NJ (US); Michael R. Cecchi, Longboat Key, FL (US)

(21) Appl. No.: 15/138,926

(22) Filed: Apr. 26, 2016

Related U.S. Application Data
(60) Provisional application No. 62/152,913, filed on Apr. 26, 2015.

(51) Int. Cl. C12M 1/00; H04N 7/18; C12M 1/34

(52) U.S. Cl. CPC C12M 41/14; C12M 41/34; H04N 7/183

(57) ABSTRACT

A temperature and gas controlled incubator for the culturing and observation of biological specimens comprising: a first wall; a second wall abutting the first wall; a third wall abutting the second wall; a top abutting the first, second and third walls; a door attached to first wall and configured to close against the third wall and top and when closed created an enclosed temperature and gas controlled volume; a first glass panel located on the first wall; a second glass panel located on the top; a third glass panel located on the third wall; a fourth glass panel located on the door; a first thin film layer located on the first glass panel, the first thin film layer configured to heat the first glass panel to a desired temperature; a second thin film layer located on the second glass panel, the second thin film layer configured to heat the second glass panel to a desired temperature; a third thin film layer located on the third glass panel, the third thin film layer configured to heat the third glass panel to a desired temperature; a fourth thin film layer located on the fourth glass panel, the fourth thin film layer configured to heat the fourth glass panel to a desired temperature; where the four thin film layers are configured to maintain a desired temperature in the enclosed temperature and gas controlled volume. A temperature and gas controlled incubator for the culturing and observation of biological specimens comprising: a first wall; a second wall abutting the first wall; a third wall abutting the second wall; a top abutting the first, second and third walls; a door attached to first wall and configured to close against the third wall and top and when closed created an enclosed temperature and gas controlled volume; a first glass panel located on the first wall; a second glass panel located on the third wall; a third glass panel abutting the first wall and third wall and forming a shelf inside the enclosed temperature and gas controlled volume, the shelf configured to hold at least one embryo culture dish; a first thin film layer located on the first glass panel, the first thin film layer configured to heat the first glass panel to a desired temperature; a second thin film layer located on the second glass panel, the second thin film layer configured to heat the second glass panel to a desired temperature; a third thin film layer located on the third glass panel, the third thin film layer configured to heat the third glass panel to a desired temperature; where the three thin film layers are configured to maintain a desired temperature in the enclosed temperature and gas controlled volume. A temperature and gas controlled incubator system for the culturing and observation of biological specimens comprising: a gas system configured to circulate, supply, and maintain proper levels of CO2, N2, and O2 to at least one temperature and gas controlled volume; a first temperature and gas controlled incubator comprising: a first wall; a second wall abutting the first wall; a third wall abutting the second wall; a top abutting the first, second and third walls; a first glass panel located on the first wall; a second glass panel located on the third wall; a first thin film layer located on the first glass panel, the first thin film layer configured to heat the first glass panel to a desired temperature; a second thin film layer located on the second glass panel, the second thin film layer configured to heat the second glass panel to a desired temperature; a door attached to first wall and configured to close against the third wall and top and when closed creates a first enclosed temperature and gas controlled volume, the first enclosed temperature and gas controlled volume in fluid communication with the gas system; a second temperature and gas controlled incubator comprising: a secondary first wall; a secondary second wall abutting the secondary first wall; a secondary third wall abutting the secondary second wall; a secondary top abutting the secondary first, secondary second and secondary third walls; a secondary first glass panel located on the secondary first wall; a secondary second glass panel located on the secondary third wall; a secondary first thin film layer located on the secondary first glass panel, the secondary first thin film layer configured to heat the secondary first glass panel to a desired temperature; a secondary second thin film layer located on the secondary second glass panel, the secondary second thin film layer configured to heat the secondary second glass panel to a desired temperature; a secondary door attached to secondary first wall and configured to close against the secondary third wall and secondary top and when closed creates a second enclosed temperature and gas controlled volume, the second enclosed temperature and gas controlled volume in fluid communication with the gas system; and where the first thin film layer and second thin film layer are configured to maintain a desired temperature in the first enclosed temperature and gas controlled volume; and where the secondary first thin film layer and secondary second thin film layer are configured to maintain a desired temperature in the second enclosed temperature and gas controlled volume. A temperature and gas controlled incubator system for the culturing and observation of biological specimens comprising: a gas system configured to circulate, supply, and maintain proper levels of CO2, N2, and O2 to at least one temperature and gas controlled volume; a first temperature and gas controlled incubator comprising: a first wall; a second wall abutting the first wall; a third wall abutting the second wall; a top abutting the first, second and third walls; a first glass panel located on the first wall; a second glass panel located on the third wall; a first heating element located on the first wall; a second heating element located on the second wall; a third heating element located on the third wall; a fourth heating element located on the top; a door attached to first wall and configured to close against the third wall and top and when closed creates a first enclosed temperature and gas controlled volume, the first enclosed temperature and gas controlled volume in fluid communication with the gas system; second temperature and gas controlled incubator comprising: a secondary first wall; a secondary second wall abutting the secondary first wall; a secondary third wall abutting the secondary second wall; a secondary top abutting the secondary first, secondary second and secondary third walls; a secondary first glass panel located on the secondary first wall; a secondary second glass panel located on the secondary third wall; a secondary first heating element located on the secondary first wall; a secondary second heating element located on the secondary second wall; a secondary third heating element located on the secondary third wall; a secondary fourth heating element located on the secondary top; a secondary door attached to secondary first wall and configured to close against the secondary third wall and secondary top and when closed creates a second enclosed temperature and gas controlled volume, the second enclosed temperature and gas controlled volume in fluid communication with the gas system; and where the first, second, third and fourth heating elements are configured to maintain a desired temperature in the first enclosed temperature and gas controlled volume; and where the secondary first, secondary second, secondary third, and secondary fourth heating elements are configured to maintain a desired temperature in the second enclosed temperature and gas controlled volume.

### TECHNICAL FIELD

This invention relates to a device which provides a controlled environment for the culturing and observation of biological specimens including embryos, oocytes and stem cells.

### BACKGROUND

Currently, there are several types of incubators for the culturing of embryos, oocytes, stem cells and other biological specimens. These incubators may have the ability to maintain temperatures levels, gas composition, and other conditions for the incubated environment.

Current incubators may come in several configurations, including a large, ‘big box’ incubator, may be made of stainless steel, may weigh up to 200 pounds, may utilize a water jacket to maintain temperature, and/or may be a direct temperature incubator where electrical coils provides the temperature. The temperature of these incubators may fluctuate internally due to the large volume of air which may be heated inconsistently by the incubator heating mechanisms. This inconsistency in the heating may relate to an inconsistency in the actual heat that is delivered to and used to maintain the proper temperature of the specimens. The specimens may be contained in a petri dish which is filled with culture medium and mineral oils.

Maintaining a proper temperature of the embryo is critical for the proper development of the embryo.

The air temperature within the ‘big box’ incubator is generally what keeps the embryo and the media it is in at the proper temperature. The temperature of the media itself may not be the desirable temperature of 37° C. and may fluctuate up and down depending on how the specimen dish (where the embryo is located) and media absorb and maintain the heat from within the ‘big box’. Current ‘big box’ incubators have exhibited inconsistencies in maintaining proper temperatures, and in maintaining the proper gas compositions.

The gases within these incubators are generally carbon dioxide, nitrogen and oxygen. The ratio of these gases may be approximately 5% carbon dioxide, 5% oxygen and 90% nitrogen. There may be other undesirable gases inside the incubator. To balance the gases within the incubator, the gases are injected into the big-box environment through a single valve which is triggered by a sensor, which may read the gases and inject either CO2 or N2, depending on the required gases and what logic determines is necessary in order to balance the environment. The problem faced in this situation is that the gases may be released into 30 Liter, 50 Liter or larger ‘big box’ incubators. The distribution of the gases may be varied throughout the big-box as they may reside in the upper portion or the lower portion and may not be distributed evenly throughout. This may cause detrimental temperature variations, and other detrimental conditions.

Currently, similar type enclosures or incubators contain metal shelves, do not directly heat these shelves, and have difficulty maintaining a consistent temperature of 37 degrees Celsius, this may be because due to the heat being supplied to ambient air within the enclosure, or heating may occur only on one end or side which has inherent problems of passing the heat evenly throughout the metal material.

An additional problem with the ‘big box’ incubator is the fact that they are built with stainless steel, which is heavy, costly, must be formed, bent and welded, take many resources, as well as a large area to build. They are heavy to ship and very costly to manufacture. The conventional incubator may weigh up to 200 pounds.

There is currently in use a second type of culturing incubator, known as ‘bench top’ incubators. These incubators a smallish in size, hold a limited number of specimen dishes, and have their own set of problems related to consistencies of gases and temperature required for the proper culturing of embryos and in maintaining the overall environment of which the embryos are held in. These incubators utilize aluminum bases to provide and maintain the heat to the holding chamber. The aluminum bases may transfer inconsistent heat due to the metals having inconsistencies of heat transfer, heat sinks, and the inability to accurately monitor the temperatures of the bases and other surfaces in the incubator.

These ‘bench top’ incubators do not always provide consistent quantity, or consistent balance of the gases that are needed in order to provide a good environment for the culturing of embryos. Most of these ‘bench top’ incubators utilize a one-way gas stream, which provides in most cases a pre-mixed percentages of the carbon dioxide, nitrogen and oxygen from a single tank source. Some of these bench top′ incubators utilize a separate gas mixing or blending system, which may allow the incubators to adjust to the amount of CO2, N2 and O2, entering the incubator. A second problem with these incubators is that they utilize a one-way gas flow system. The gases enter the system at one end of the chamber, move through the chamber and are then expelled from the opposite end of the chamber, into the outside environment, or the laboratory, repeatedly. This method may use up more gas than is necessary or needed at any particular time and deplete the gas tanks, or require greater amounts of gas. The use of a pre-mixed gas tank may also become less effective over time, in that the gases may separate within the tank and may provide the bench top with unknown percentages of each of the gases. An additional problem facing a bench top incubator is the inconsistent temperature of the heated surface during the incubation stages. This is due to the using a metal surfaces, which is heated, which may transfer inconsistent heating to the culturing dishes that are placed on the surface. The possibility of varying temperature and various heat sinks of the device may create problematic varying temperature on the metal surface.

Other problems facing the traditional incubators and bench top incubators is that they do not recirculate the gases, and therefore rely on the original gas mix or the gas being delivered to the system for the correct percentage of the CO2, N2 and O2. The ‘big box’ incubators simple dump portions of gases into the environment, where it may not properly blend and may not be homogenous throughout the incubator.

An additional problem with the ‘big box’ incubators is that they generally do not provide for any gas exchanges, electrical connectors, internet, computer terminals and connections, within the box itself. This limits the ability to obtain information of the specimens within, does not allow secondary equipment to be easily installed and connected such as microscopes, small incubation systems, imaging devices or diagnostic device.

Thus there is a need for an invention that overcomes the above listed and other disadvantages.

### SUMMARY OF THE INVENTION

The invention relates to a temperature and gas controlled incubator for the culturing and observation of biological specimens comprising: a first wall; a second wall abutting the first wall; a third wall abutting the second wall; a top abutting the first, second and third walls; a door attached to first wall and configured to close against the third wall and top and when closed created an enclosed temperature and gas controlled volume; a first glass panel located on the first wall; a second glass panel located on the top; a third glass panel located on the third wall; a fourth glass panel located on the door; a first thin film layer located on the first glass panel, the first thin film layer configured to heat the first glass panel to a desired temperature; a second thin film layer located on the second glass panel, the second thin film layer configured to heat the second glass panel to a desired temperature; a third thin film layer located on the third glass panel, the third thin film layer configured to heat the third glass panel to a desired temperature; a fourth thin film layer located on the fourth glass panel, the fourth thin film layer configured to heat the fourth glass panel to a desired temperature; where the four thin film layers are configured to maintain a desired temperature in the enclosed temperature and gas controlled volume.

The invention also relates to a temperature and gas controlled incubator for the culturing and observation of biological specimens comprising: a first wall; a second wall abutting the first wall; a third wall abutting the second wall; a top abutting the first, second and third walls; a door attached to first wall and configured to close against the third wall and top and when closed created an enclosed temperature and gas controlled volume; a first glass panel located on the first wall; a second glass panel located on the third wall; a third glass panel abutting the first wall and third wall and forming a shelf inside the enclosed temperature and gas controlled volume, the shelf configured to hold at least one embryo culture dish; a first thin film layer located on the first glass panel, the first thin film layer configured to heat the first glass panel to a desired temperature; a second thin film layer located on the second glass panel, the second thin film layer configured to heat the second glass panel to a desired temperature; a third thin film layer located on the third glass panel, the third thin film layer configured to heat the third glass panel to a desired temperature; where the three thin film layers are configured to maintain a desired temperature in the enclosed temperature and gas controlled volume.

In addition, the invention relates to a temperature and gas controlled incubator system for the culturing and observation of biological specimens comprising: a gas system configured to circulate, supply, and maintain proper levels of CO2, N2, and O2 to at least one temperature and gas controlled volume; a first temperature and gas controlled incubator comprising: a first wall; a second wall abutting the first wall; a third wall abutting the second wall; a top abutting the first, second and third walls; a first glass panel located on the first wall; a second glass panel located on the third wall; a first thin film layer located on the first glass panel, the first thin film layer configured to heat the first glass panel to a desired temperature; a second thin film layer located on the second glass panel, the second thin film layer configured to heat the second glass panel to a desired temperature; a door attached to first wall and configured to close against the third wall and top and when closed creates a first enclosed temperature and gas controlled volume, the first enclosed temperature and gas controlled volume in fluid communication with the gas system; a second temperature and gas controlled incubator comprising: a secondary first wall; a secondary second wall abutting the secondary first wall; a secondary third wall abutting the secondary second wall; a secondary top abutting the secondary first, secondary second and secondary third walls; a secondary first glass panel located on the secondary first wall; a secondary second glass panel located on the secondary third wall; a secondary first thin film layer located on the secondary first glass panel, the secondary first thin film layer configured to heat the secondary first glass panel to a desired temperature; a secondary second thin film layer located on the secondary second glass panel, the secondary second thin film layer configured to heat the secondary second glass panel to a desired temperature; a secondary door attached to secondary first wall and configured to close against the secondary third wall and secondary top and when closed creates a second enclosed temperature and gas controlled volume, the second enclosed temperature and gas controlled volume in fluid communication with the gas system; and where the first thin film layer and second thin film layer are configured to maintain a desired temperature in the first enclosed temperature and gas controlled volume; and where the secondary first thin film layer and secondary second thin film layer are configured to maintain a desired temperature in the second enclosed temperature and gas controlled volume.

The invention also relates to a temperature and gas controlled incubator system for the culturing and observation of biological specimens comprising: a gas system configured to circulate, supply, and maintain proper levels of CO2, N2, and O2 to at least one temperature and gas controlled volume; a first temperature and gas controlled incubator comprising: a first wall; a second wall abutting the first wall; a third wall abutting the second wall; a top abutting the first, second and third walls; a first glass panel located on the first wall; a second glass panel located on the third wall; a first heating element located on the first wall; a second heating element located on the second wall; a third heating element located on the third wall; a fourth heating element located on the top; a door attached to first wall and configured to close against the third wall and top and when closed creates a first enclosed temperature and gas controlled volume, the first enclosed temperature and gas controlled volume in fluid communication with the gas system; second temperature and gas controlled incubator comprising: a secondary first wall; a secondary second wall abutting the secondary first wall; a secondary third wall abutting the secondary second wall; a secondary top abutting the secondary first, secondary second and secondary third walls; a secondary first glass panel located on the secondary first wall; a secondary second glass panel located on the secondary third wall; a secondary first heating element located on the secondary first wall; a secondary second heating element located on the secondary second wall; a secondary third heating element located on the secondary third wall; a secondary fourth heating element located on the secondary top; a secondary door attached to secondary first wall and configured to close against the secondary third wall and secondary top and when closed creates a second enclosed temperature and gas controlled volume, the second enclosed temperature and gas controlled volume in fluid communication with the gas system; and where the first, second, third and fourth heating elements are configured to maintain a desired temperature in the first enclosed temperature and gas controlled volume; and where the secondary first, secondary second, secondary third, and secondary fourth heating elements are configured to maintain a desired temperature in the second enclosed temperature and gas controlled volume.

### BRIEF DESCRIPTION OF THE DRAWINGS

The present disclosure may be better understood by those skilled in the pertinent art by referencing the accompanying drawings, where like elements are numbered alike in the several figures, in which:

FIG. 1 is a perspective view of the invention;

FIG. 2 is a perspective view of another embodiment of the invention;

FIG. 3 is a schematic view of another embodiment of the invention;

FIG. 4 is an exploded view of an embodiment of the invention;

FIG. 5 is a cross-sectional view of an embodiment of the invention;

FIG. 6 is a cross-sectional view of another embodiment of the invention;

FIG. 7 a cross-sectional view of an embodiment of the invention with 4 chambers;

FIG. 8 is a schematic of another embodiment of the invention;

FIG. 9 is a top view of the treated glass;

FIG. 10 is a front view of the treated glass;

FIG. 11 is a top view showing four areas of thin film on treated glass; and

FIG. 12 shows a top view of three areas of thin film on treated glass.

### DETAILED DESCRIPTION

The disclosed device can provide a more accurate controlled temperature for a controlled environment, a more compact gas system for the culturing of biological specimens such as embryos, oocytes, stem cells and other cells. The disclosed system can monitor those specimens and provide displays for users to see important information regarding the specimens and the device.

The invention may comprise a treated glass system to maintain a more precise temperature level of the interior of the incubator. The interior surfaces may be configured to be about 37° C. at all times. The treated glass systems may comprise a glass, which has been coated with a thin-film material that can heat the glass. The thin-film material may be attached to an electrical heating source via electrical wires. The heating source may communicate heat via the wires to the glass, and maintain the proper temperature of the glass. Thermo-Stone, Reno, Nev., USA may provide such a thin-film The thin-film on the glass for this invention is located in the area where a consistent monitored temperature is desired or to provide heat to the overall interior. This may be an overall large piece of glass, which may be used to heat an overall environment, or the areas where the culturing dishes or devices may be sitting on. This thin-filmed glass system is able to maintain about 37° C., even with a large displacement of air and a number of dishes in contact with the glass. This glass will allow the invention to maintain a surface temperature for the culture dishes to reside on and allow the embryos within these dishes to be observed from below by a camera system.

In one of the embodiments the glass surface may have the film in the middle portion of the glass heated independently to maintain 37° C. The glass may contain a second thin-film application to the outer portion of the glass, not in contact with the middle portion of thin film. This may allow the glass to perform at least two separate heating functions. The outer portion application may be used to keep the glass from losing its temperature in this outer portion, avoiding a heat sink. This outer portion may be used to heat the chamber walls, which the dishes and embryos reside in and to heat the ambient air within this chamber.

The thin film glass may be transparent and may be used as a top, cover or lid to the chambers, help to maintain the heat and allows the user to observe the specimens through this glass, without opening the cover or lid.

The thin-film material allows for the glass to have a consistent temperature. The thin-film material is lightweight. The thin-film material can operate for long periods of time without replacement or service issues. The use of glass components for the heating source greatly reduces the cost of production and the cost of operation.

The glass inserts of the invention may be heated by a heat source provided at the edges of the glass, which will then heat the entire glass and its surface throughout. The temperature may be repeatedly monitored and adjusted to maintain the correct heat for the culture dishes, which reside on the glass, which should be about 37 degrees, Celsius.

In one of the embodiment the invention may include a temperature controlled incubation area, a lid to the incubation area, the thin-film glass as the surface for the petri dishes to sit on, a camera system to take images, and a controlled recirculation of the gases. The systems may be monitored through a controller and sensors, which may be attached to a touch screen, display screen, and/or a series of control buttons and displays.

In a second embodiment of the invention the incubator may consist of a multiple number of the thin-film applications shelves, walls, base and ceiling. This would allow a very efficient method of temperature control, similar to a big-box incubator and may weigh less than 50 pounds. It would not require a cumbersome water jacket method or direct heat method which are currently used in some big-box incubators. The advantages would be to precisely control the interior temperature as well as the temperature of each shelf. This may allow a greatly improved environment for the specimens for the culturing, growth and ultimately a positive outcome for the embryos cultured in this invention.

An additional embodiment of the invention is using a heated glass surface to maintain a consistent temperature for the culture dish to reside on, and have a camera system to take images of the embryos, from underneath the glass insert. This embodiment utilized the effective gas circulating system, temperature monitoring and the superior gas environment created in the chamber, which is common through the embodiments.

An additional embodiment is having a chamber with a solid bottom, which is temperature controlled, for the dishes, holding the embryos, to reside on. The solid bottom allows the invention to be used as an embryo culture incubator, utilizing this technology to heat and maintain the surface and to utilize the recirculating gas system to create a better environment for embryo development and outcome.

An additional embodiment is to assemble multiple chambers, in line or in a pattern, within a single enclosure, utilizing a single gas circulation system to provide the balance of gases with in each chamber. Their may be about 4 to about 10 chambers assembled generally together and generally inline, depending on the intended use.

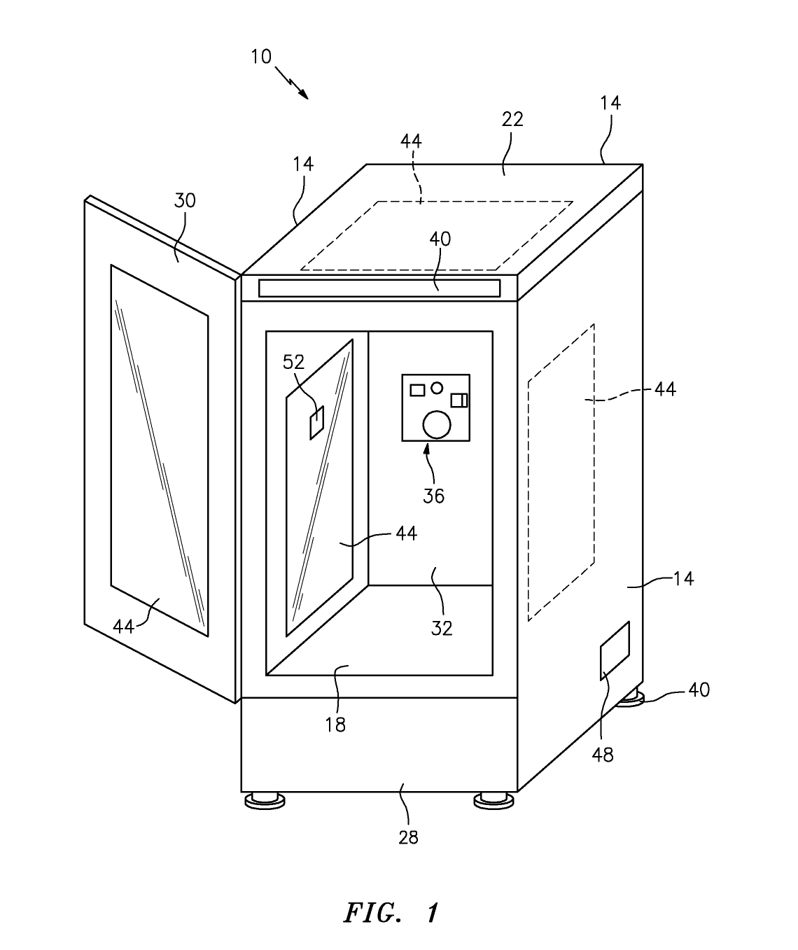

FIG. 1 illustrates one of the embodiments of the invention. The image shows the device 10, an incubator enclosure system for the culturing of embryos. The device 10 consists of outer walls 14, a base 18, a top 22, rear wall 32, door 30, lower portion 28, and a set of feet 40, a display area 42. In this embodiment the invention contains several treated glass panels 44.

The glass panels 44 may provide heat to the interior of the incubator and may maintain a temperature of about 37 degrees Celsius. The glass panels 44 may be temperature controlled, and comprise thin-film technology placed on at least one side of the glass panel, which is heated, by the use of a low electrical voltage of from 12 to 125 volts. Voltages between about 12 and about 24 volts may be preferably used because the power sources are readily available and they generally use less power and save money.

The walls 14, top 22, bottom 28, door 30 and rear 32, may contain an insulation material and outer wall encasement to help maintain the temperature within. The rear wall 32 may be made of a material such as stainless steel, metal or plastic. The rear wall 32 may comprise various pass thru holes for items such as connectors for WIFI, imaging, power, monitoring and the like. These connectors may allow secondary device such s sensor, controller or various pieces of equipment to be placed within the enclosure and be able to be operated, connected through the rear wall to eternal communications and displays.

The rear wall may comprise a connection port 36, which may allow gases to be introduced in the device 10. These gases CO2, N2 and O2 are introduced into the enclosure to give the embryos or other specimens the correct amount and balance of these gases in order to properly grow and develop.

The port 36, may allow the recirculating of the gases, and the connection of electrical, imaging, USB, internet and the attachment of secondary equipment in and out of this invention.

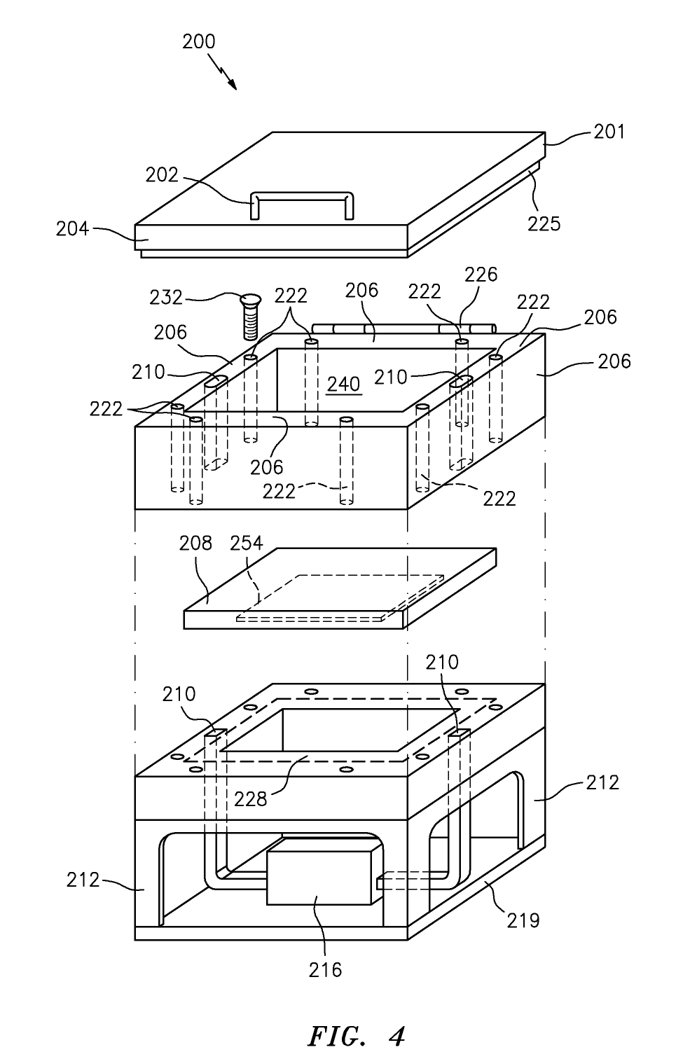

The gasses may be provided to the invention through an onboard system, which may be held in the lower portion 28. This lower portion 28 may contain a gas system, which may be similar to the configuration shown in FIG. 4, or an external circulating gas system, which may be a stand-alone unit, such as a gas mixture and recirculation components. This on-board gas system will take the incoming gases, circulate these gases through the incubator portion and then through a sensor device, which monitors this ongoing gas circulation with a series of sensors to read the gas levels in this gas stream, then to open certain solenoids to allow certain gases to enter the system, such as CO2, N2 or O2. The system will be able to closely maintain the required gas mixes of about 5% CO2, N2 and O2.

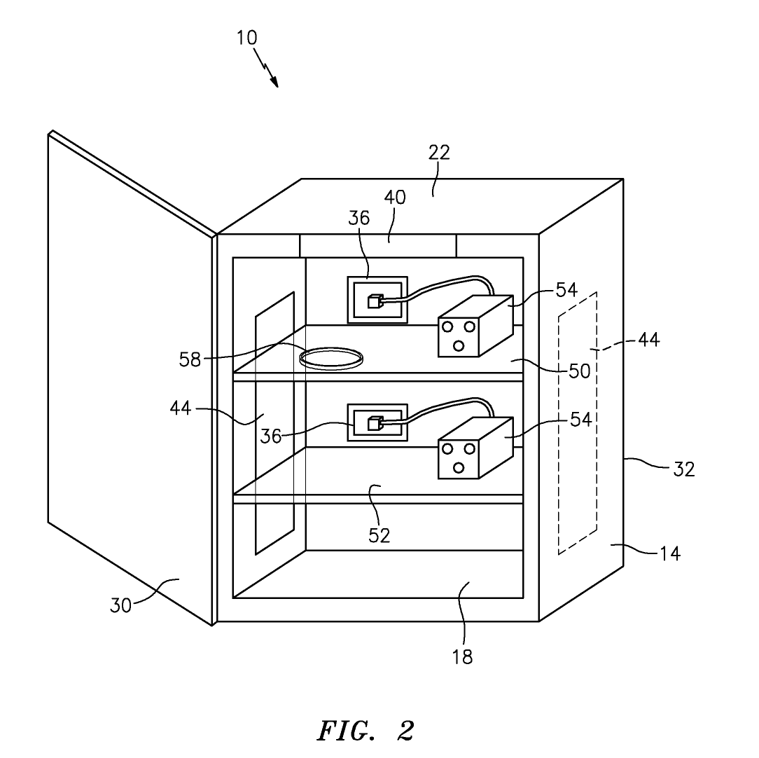

FIG. 2, Shows an additional embodiment of the invention. This embodiment comprises thin-film treated glass shelves to better control the temperature of the shelf and any dishes or devices placed on top of these shelves. These shelves are made of a treated glass, which are coated with a thin-film conductive material, which allows the direct and controlled heating of these shelves. In this embodiment the embryo culture dishes 58 are supported by treated glass shelves 50, 52. The shelves 50, 52 may also be used to support secondary devices 54, which may be used to hold a device which may hold the embryos, such as large platter or a small camera unit which may be use to observe embryos. The invention supplies these devices with a ‘circulating’ gas mixer and better gas balance and desired concentration, unlike conventional units.

The shelves 50, 52 may allow the placement of several embryo culture dishes and several secondary devices 54. This embodiment gives better temperature control of the shelves, which the embryo culture dishes reside on and supplies those dishes with a more accurate concentration of gases within the environment.

The shelves in these embodiments may be made of a temperature maintaining material, such as stainless steel or aluminum, or a copolymer. These shelves may be heated from below or not and may draw their heat from the ambient heated air of the interior of the incubator. These shelve options allow the reduction of materials, weight and cost to construct.

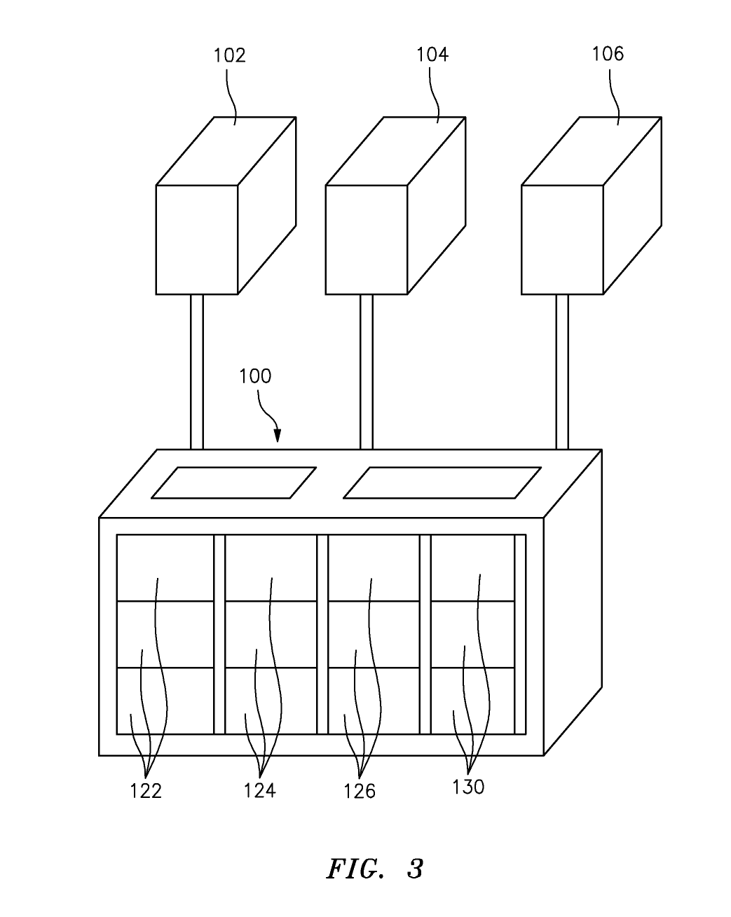

FIG. 3 shows an additional embodiment of the invention. In this embodiment, several devices are attached to a single central device, which control and distributes the gases, monitors the temperature, imaging and additional desired tasks. The central device 100 contains a gas mixing system, which may provide the correct amount of gases, CO2, N2 and O2 to the series of units, shown as 102, 104 and 106. These units 102, 104, 106 may be variations of the invention 10 shown in FIGS. 1-2. The central unit 100 may monitor and display the percentages of the gases 122, within the system, and to display the temperature 124, images 126 and controls 130. This display screen may be a touch screen and similar interactive technologies.

An advantage to this component 100, is that it may allow the multiple devices 102, 104, 106 to attach to this central component 100, which allows maintaining a single controlled gases, temperature control system throughout these devices, and allows for observation of the biological specimens, resulting in a very effective, diversified system which may be overall less expensive to purchase and maintain and is superior to current incubator systems.

FIG. 4 shows an exploded view of an embodiment of the temperature and gas controlled incubated environment. This embodiment utilizes the thin-film treated glass technology to precisely control the temperature of the surface the embryo culture dish may reside on and with a second thin-film application to control the temperature of the environment through the transfer of heat into the exterior walls of the incubator section of the invention. The heating of the walls, may maintain the heat of the incubated environment. Both of the areas of treated surface and heat control can be operated from a single device. The device 200, consists of a cover 201, handle 202, insulation 204 (not shown) inside the cover 201, a chamber 240 surrounded by chamber walls 240, thin-filmed glass 208, lower support 212, and base 219. The gas system 216 is contained in the lower portion of the device 200. The gas from the system 216 may be delivered to the chamber 240 through channels 210 within the walls 206 of the chamber 240. The chamber walls 206 are secured to the lower support 212, by a series of securing devices, such as rivets or screws 222. The chamber 240 may range in size, from about 4 to 20 inches, square, and about 3 inches in height, or any other suitable size. The glass 208, may sit on the support 212, within the area identified as 228. This may allow the chamber 240 to sit on top of the support 212 and the glass 208.

The gas system 216 continually circulates the gases through, a system of gas sensors and solenoids to measure the gases with sensors and then to open and close the solenoids to repeatedly add additional gases to maintain a gas mixture of about 5% carbon dioxide (CO2), about 5% oxygen (O2) and about 90% nitrogen (N2).

The incubated chamber 240 may be of a single construction. This part may be made by including the sidewall and base in a formed part, which may be a ‘punch out’ or a single part, possibly aluminum with the chamber portion honed out of the aluminum part. This allows certain benefits for production and temperature controls, as well as gas circulation. This embodiment allows the invention to have a single part, without glass components at the base, whereby the embodiments will not take images.

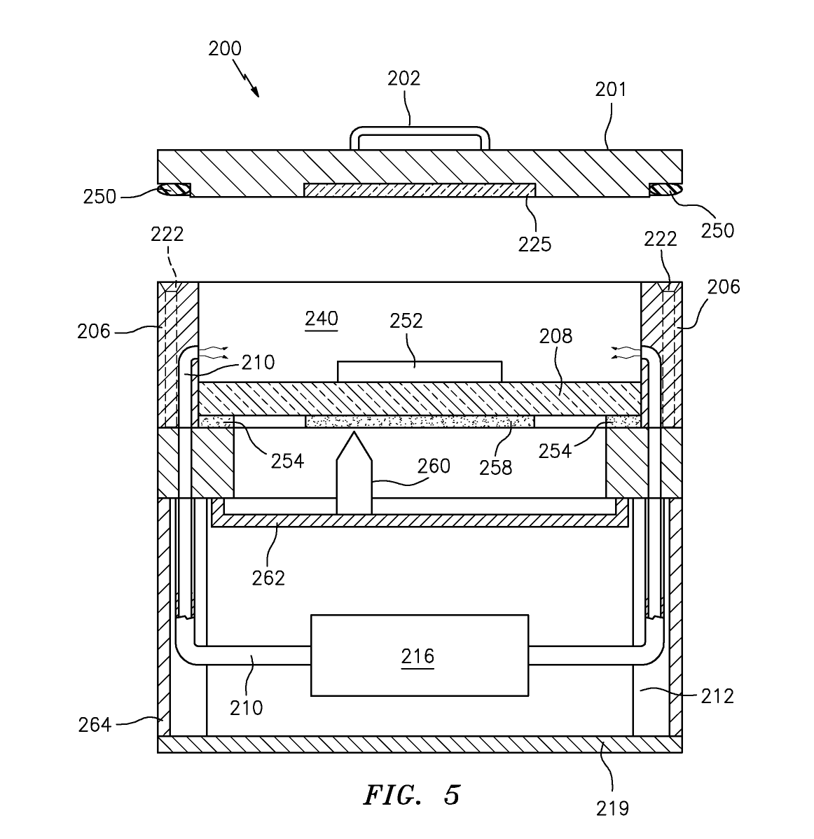

FIG. 5 shows a front view cross-sectional view of another embodiment of the temperature and gas controlled incubator. This embodiment creates a closed incubated control environment and a recirculating gas system for the incubation, growth, and development and of embryos and additional specimens. The components are the cover 201, the chamber walls 206, the chamber 240, a glass 208, support 212, the box base 219, gas system 216 and its components. This embodiment may be temperature controlled similar to that of FIG. 4. This embodiment includes a camera system 260, which is mounted on support brackets 262. The bracket allows the camera to move in three dimensions, sideways and up and down for focusing capabilities. This camera system will be able to precise pictures of the embryos, within the dish 252 and to transmit theses images to a system that will record, save and display these images at the request of the operator. The incubator may include a light source 225.

FIG. 6 shows shows a front view cross-sectional view of another embodiment of the temperature and gas controlled incubator. This embodiment creates a closed incubated control environment and a recirculating gas system for the incubation, growth, development and of embryos and additional specimens. The components are the cover 201, the chamber walls 206, the chamber 240, a solid bottom 209, support 212, the box base 219, gas system 216. This embodiment has a solid base 209, which may be made of aluminum or stainless steel. The chamber may be of multiple parts or may be of a single construction. This embodiment contains a gas recirculating gas-mixing system 216 in the lower portion, where the mixed gases enter the chamber 240 through a gas port 217 and exits the chamber 240 through a return port 218, through the connecting tubing 210. This allows a recirculating gas loop between the gas mixer 216 and the chamber 240. This will maintain the desired levels of CO2, N2 and O2. This embodiment allows a device 200, which allow closely controlling the temperature and gas mix, creating a controlled system, better embryo development and the possibility of ore live birth. In one embodiment, the incubator described in FIG. 6 may be assembled without the use of screws. The advantages to the system is that it may be of a simpler construction, may be able to maintain temperatures more accurately and will result in better embryo development and results in more live births.

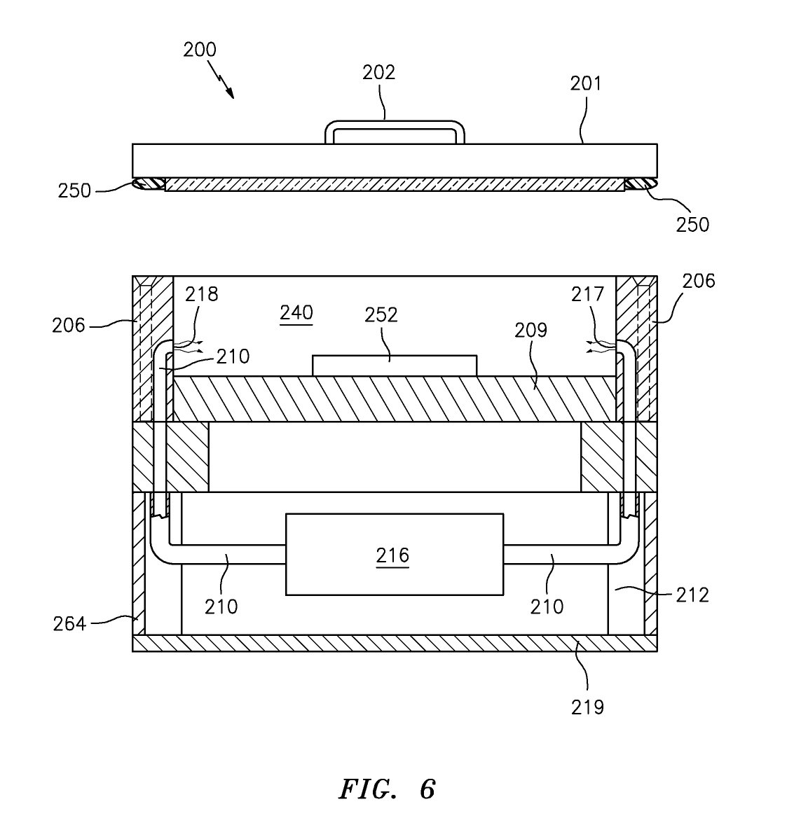

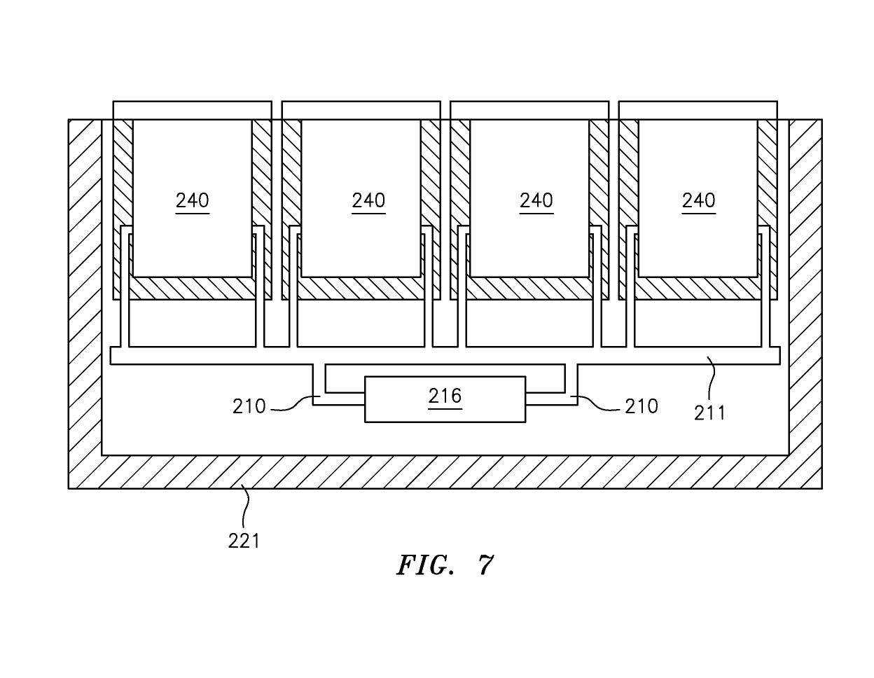

FIG. 7 shows another embodiment of the invention. In this embodiment there are multiple chambers 240. The chambers 240 in FIG. 7 are similar to the chambers in FIG. 6 and are heated, monitored and controlled in a similar fashion. The multiple chambers 240, in this embodiment may be placed side by side in an outer enclosure 221, which may hold and help to maintain the chambers' environments. The chambers 240 may be supplied with gases through a manifold 211, which introduces gas to the chambers 240 and has a return connection. The manifold 211 allows the continuous circulation of the gases as well as the repeated monitoring and adjustments of those gases though the gas mixer 216. The operations and monitoring information may be then displayed on a display screen or be sent to an additional device. This embodiment allows multiple chambers 240 to be supported by a single gas mixer 216. As the chambers 240 are independently controlled, this embodiments may be simply and effectively expanded to 6, 8 or more chambers with a single enclosure 221 and utilizing a single gas mixers, which will allow expansion and cost savings, while maintaining an effective embryo development system.

FIG. 8 shows a diagram of the gas mixer 216, shown in FIGS. 4, 5, 6 and 7. The mixer 216 is fed by incoming gas lines for carbon dioxide 300, Nitrogen 301, Oxygen 302. The gases may be drawn from the ambient air. The incoming a gases will enter the system through sensors, 310 which will open and close to allow the sensors 320, 321, 322 to adjust the gas mixture.

The gas mixer is a continuous loop, the gas moves from the mixing box 305 to the devices 350, which may be an incubator chamber as described in FIGS. 4, 5 6, and 7 or other compatible devices. The gas returns to the mixer from the devices 350 and enter a mix box 306, which contains sensors. These sensors 320, 321 and 322 monitor the gas levels for the gas components CO2, O2, and N2, then report these readings to a controller, not shown, which will then open and close the appropriate solenoid, either CO2, N2 or O2 to create the desired gas levels of the user. These gas levels should be about 5% CO2, about 95% N2 and about 5% O2, for embryonic development. The gases then move through a filter 340 and pump 345, which pushes the gases through the system. The filter 340 is used to filter our particulates and airborne contaminants.

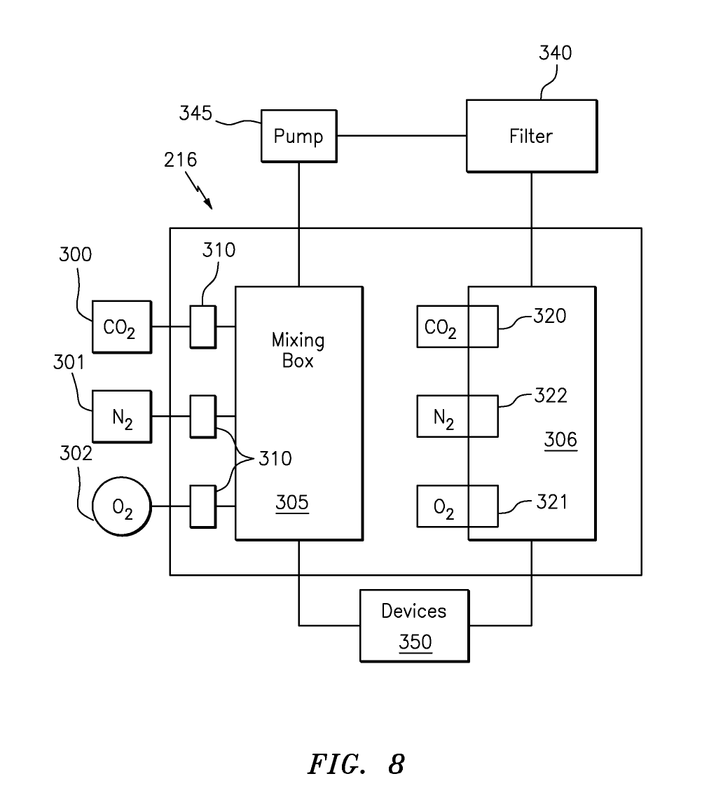

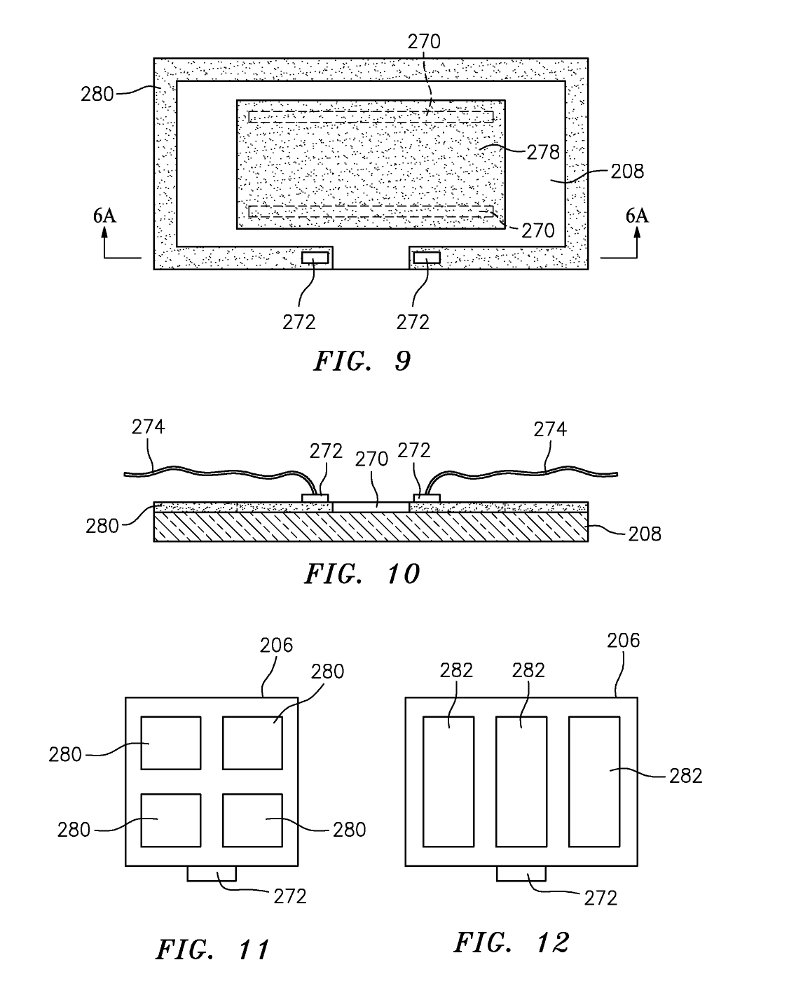

FIGS. 9 and 10 show an example of the treated glass and connection to be used in the invention. The image of from the underside of the treated glass 208, showing the areas of treatment 278, 280 and the wire connectors 270. The glass 208, contains an inner treated area 278, with two wire connectors 270, which may be connected to the power and monitoring source (not shown), and an outer film 280, which is on the perimeter of the glass, and connected by wire connectors 272 to independent wires 274, connected to a power source (not shown).

FIGS. 11 and 12 illustrate additional thin film and heat patterns for a treated glass 206, which may apply to the invention. FIG. 6B shows a four panel film configurations, where each of the quadrants 280 may be independently heated, controlled separately and be of different temperatures, without interfering with each other. FIG. 6C shows a treated glass 206 with three panels 282, where each of the panels 282 may be independently heated, controlled separately and be of different temperature.

The disclosed gas circulation system has many advantages. The device can control the temperature of the total incubated environment and to be able control the temperature of the surface or shelves which the specimen dishes are placed upon, and to ultimately control the temperatures of the dishes and the culture medium in the dish of which the embryo is residing in. The device can provide the proper best balance of gases, namely CO2, N2 and O2 and to be able to recirculate and adjust the concentration of the gases on a repeated basis. The device can better control the overall temperature of the chamber of which the embryos are placed in to be more conducive to the culturing, survival, outcome and a greater likelihood of live birth. The device can better control the temperature of the environment, concentration of the gases and to allow internal ports for the external connections of the electrical, imaging, observation and monitoring devices.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it may be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure may include all embodiments falling within the scope of the appended claims.

What is claimed is:

1. A temperature and gas controlled incubator for the culturing and observation of biological specimens comprising:
a first wall;
a second wall abutting the first wall;
a third wall abutting the second wall;
a top abutting the first, second and third walls
a door attached to first wall and configured to close against the third wall and top and when closed created an enclosed temperature and gas controlled volume;
a first glass panel located on the first wall;
a second glass panel located on the top;
a third glass panel located on the third wall;
a fourth glass panel located on the door;
a first thin film layer located on the first glass panel, the first thin film layer configured to heat the first glass panel to a desired temperature;
a second thin film layer located on the second glass panel, the second thin film layer configured to heat the second glass panel to a desired temperature;
a third thin film layer located on the third glass panel, the third thin film layer configured to heat the third glass panel to a desired temperature;
a fourth thin film layer located on the fourth glass panel, the fourth thin film layer configured to heat the fourth glass panel to a desired temperature; and
wherein the four thin film layers are configured to maintain a desired temperature in the enclosed temperature and gas controlled volume.

2. The temperature and gas controlled incubator of claim 1, wherein each of the thin film layers are configured to receive a voltage of about 12 volts to about 24 volts.

3. The temperature and gas controlled incubator of claim 1, further comprising:
an insulating material located in the first wall, second wall, third wall, top, and top.

4. The temperature and gas controlled incubator of claim 1, further comprising:
a connection port in one of the walls, where the connection port is configured to circulate CO2, N2, and O2 gases into the temperature and gas controlled volume.

5. The temperature and gas controlled incubator of claim 1, further comprising a gas system in fluid communication with the temperature and gas controlled volume and configured to circulate, supply, and maintain proper levels of CO2, N2, and O2 to the temperature and gas controlled volume.

6. A temperature and gas controlled incubator for the culturing and observation of biological specimens comprising:
a first wall;
a second wall abutting the first wall;
a third wall abutting the second wall;
a top abutting the first, second and third walls a door attached to first wall and configured to close against the third wall and top and when closed created an enclosed temperature and gas controlled volume;
a first glass panel located on the first wall;
a second glass panel located on the third wall;
a third glass panel abutting the first wall and third wall and forming a shelf inside the enclosed temperature and gas controlled volume, the shelf configured to hold at least one embryo culture dish;
a first thin film layer located on the first glass panel, the first thin film layer configured to heat the first glass panel to a desired temperature;
a second thin film layer located on the second glass panel, the second thin film layer configured to heat the second glass panel to a desired temperature;
a third thin film layer located on the third glass panel, the third thin film layer configured to heat the third glass panel to a desired temperature;
wherein the three thin film layers are configured to maintain a desired temperature in the enclosed temperature and gas controlled volume.

7. The temperature and gas controlled incubator of claim 6, wherein the shelf is further configured to hold at least one secondary device configured to hold and observe embryos placed within or on top of the incubator.

8. A temperature and gas controlled incubator system for the culturing and observation of biological specimens comprising:
a gas system configured to circulate, supply, and maintain proper levels of CO2, N2, and O2 to at least one temperature and gas controlled volume;
a first temperature and gas controlled incubator comprising:
a first wall;
a second wall abutting the first wall;
a third wall abutting the second wall;
a top abutting the first, second and third walls;
a first glass panel located on the first wall;
a second glass panel located on the third wall;
a first thin film layer located on the first glass panel, the first thin film layer configured to heat the first glass panel to a desired temperature;
a second thin film layer located on the second glass panel, the second thin film layer configured to heat the second glass panel to a desired temperature;
a door attached to first wall and configured to close against the third wall and top and when closed creates a first enclosed temperature and gas controlled volume, the first enclosed temperature and gas controlled volume in fluid communication with the gas system;
a second temperature and gas controlled incubator comprising:
a secondary first wall;
a secondary second wall abutting the secondary first wall;
a secondary third wall abutting the secondary second wall;
a secondary top abutting the secondary first, secondary second and secondary third walls;
a secondary first glass panel located on the secondary first wall;
a secondary second glass panel located on the secondary third wall;
a secondary first thin film layer located on the secondary first glass panel, the secondary first thin film layer configured to heat the secondary first glass panel to a desired temperature;
a secondary second thin film layer located on the secondary second glass panel, the secondary second thin film layer configured to heat the secondary second glass panel to a desired temperature;
a secondary door attached to secondary first wall and configured to close against the secondary third wall and secondary top and when closed creates a second enclosed temperature and gas controlled volume, the second enclosed temperature and gas controlled volume in fluid communication with the gas system; and
wherein the first thin film layer and second thin film layer are configured to maintain a desired temperature in the first enclosed temperature and gas controlled volume; and wherein the secondary first thin film layer and secondary second thin film layer are configured to maintain a desired temperature in the second enclosed temperature and gas controlled volume.

9. The temperature and gas controlled incubator system of claim 8, further comprising:
a third glass panel located on the top;
a fourth glass panel located on the door;
a third thin film layer located on the third glass panel, the third thin film layer configured to heat the third glass panel to a desired temperature;
a fourth thin film layer located on the fourth glass panel, the fourth thin film layer configured to heat the fourth glass panel to a desired temperature;
a secondary third glass panel located on the secondary top;
a secondary fourth glass panel located on the secondary door;
a secondary third thin film layer located on the secondary third glass panel, the secondary third thin film layer configured to heat the secondary third glass panel to a desired temperature;
a secondary fourth thin film layer located on the secondary fourth glass panel, the secondary fourth thin film layer configured to heat the secondary fourth glass panel to a desired temperature.

10. The temperature and gas controlled incubator system of claim 8, further comprising:
a third glass panel abutting the first wall and third wall and forming a shelf inside the enclosed temperature and gas controlled volume, the shelf configured to hold at least one embryo culture dish;
a third thin film layer located on the third glass panel, the third thin film layer configured to heat the third glass panel to a desired temperature;
a secondary third glass panel abutting the secondary first wall and secondary third wall and forming a secondary shelf inside the second enclosed temperature and gas controlled volume, the secondary shelf configured to hold at least one embryo culture dish;
a secondary third thin film layer located on the secondary third glass panel, the secondary third thin film layer configured to heat the secondary third glass panel to a desired temperature.

11. The temperature and gas controlled incubator system of claim 8, further comprising a camera system in optical communication with at least one of the glass panels and configured to capture images inside at least one of the incubators.

12. A temperature and gas controlled incubator system for the culturing and observation of biological specimens comprising:
a gas system configured to circulate, supply, and maintain proper levels of CO2, N2, and O2 to at least one temperature and gas controlled volume;
a first temperature and gas controlled incubator comprising:
a first wall;
a second wall abutting the first wall;
a third wall abutting the second wall;
a top abutting the first, second and third walls;
a first glass panel located on the first wall;
a second glass panel located on the third wall;
a first heating element located on the first wall;
a second heating element located on the second wall;
a third heating element located on the third wall;
a fourth heating element located on the top;
a door attached to first wall and configured to close against the third wall and top and when closed creates a first enclosed temperature and gas controlled volume, the first enclosed temperature and gas controlled volume in fluid communication with the gas system;
a second temperature and gas controlled incubator comprising:
a secondary first wall;
a secondary second wall abutting the secondary first wall;
a secondary third wall abutting the secondary second wall;
a secondary top abutting the secondary first, secondary second and secondary third walls;
a secondary first glass panel located on the secondary first wall;
a secondary second glass panel located on the secondary third wall;
a secondary first heating element located on the secondary first wall;
a secondary second heating element located on the secondary second wall;
a secondary third heating element located on the secondary third wall;
a secondary fourth heating element located on the secondary top;
a secondary door attached to secondary first wall and configured to close against the secondary third wall and secondary top and when closed creates a second enclosed temperature and gas controlled volume, the second enclosed temperature and gas controlled volume in fluid communication with the gas system; and
wherein the first, second, third and fourth heating elements are configured to maintain a desired temperature in the first enclosed temperature and gas controlled volume; and wherein the secondary first, secondary second, secondary third, and secondary fourth heating elements are configured to maintain a desired temperature in the second enclosed temperature and gas controlled volume.

13. The temperature and gas controlled incubator system of claim 12, further comprising a camera system in optical communication with at least one of the glass panels and configured to capture images inside at least one of the incubators.

14. The temperature and gas controlled incubator system of claim 12, comprising plurality of additional enclosed temperature and gas control volumes in series or parallel fluid communication with each other, and each additional volume in fluid communication with the gas system, and the gas system is further configured to circulate, supply, and maintain proper levels of CO2, N2, and O2 to each of the additional volumes.

15. The temperature and gas controlled incubator system of claim 14, comprising at least a total of 10 enclosed temperature and gas control volumes.
